(19) United States
(12) Patent Application Publication
(10) Pub. No.: US 2023/0044802 A1
(43) Pub. Date: Feb. 9, 2023

(54) SYSTEMS AND METHODS FOR AN AUTOMATED MATCHING SYSTEM FOR HEALTHCARE PROVIDERS AND REQUESTS

(71) Applicants: Michael Coen, Pittsburgh, PA (US); Manoj Kamavarapu, Pittsburgh, PA (US); Timothy Schruben, Pittsburgh, PA (US)

(72) Inventors: Michael Coen, Pittsburgh, PA (US); Manoj Kamavarapu, Pittsburgh, PA (US); Timothy Schruben, Pittsburgh, PA (US)

(21) Appl. No.: 17/787,450

(22) PCT Filed: Dec. 21, 2020
(86) PCT No.: PCT/US20/66391

§ 371 Date: Jun. 20, 2022

Related U.S. Application Data
(60) Provisional application No. 62/953,161, filed on Dec. 23, 2019.

(51) Int. Cl. G06Q 40/08; G06Q 30/06

(52) U.S. Cl. CPC G06Q 40/08; G06Q 30/0611

(57) ABSTRACT

One embodiment provides a system for automatically generating and ranking search results matching a healthcare request, the system including: instructions to: retrieve, from at least one of a consumer device or a first provider device, one or more requests, each request associated with a healthcare service and including at least one criterion associated with the service; perform at least one of: requesting one or more bids from one or more second provider devices or retrieving one or more stored bids from a database of bids, the one or more requested or retrieved bids matching the healthcare service of the one or more requests; using a recommendation engine and the at least one criterion, generate a ranking of the one or more requested or retrieved bids; generate a graphical user interface including the one or more requested or retrieved bids; and output the graphical user interface.

### CROSS REFERENCE TO RELATED APPLICATIONS

This application is a PCT application of co-pending U.S. Provisional Patent Application Ser. No. 62/953,161, entitled “SYSTEMS AND METHOD FOR AN AUTOMATED MATCHING SYSTEM FOR HEALTHCARE PROVIDERS AND REQUESTS,” and filed on Dec. 23, 2019, the contents of which is hereby incorporated by reference in its entirety.

### TECHNICAL FIELD

The present disclosure related generally to the fields of searching and matching algorithms and interactive graphical user interfaces. More specifically, and without limitation, this disclosure relates to systems and methods for automatically generating and ranking search results matching a healthcare request, and generating associated graphical user interfaces.

### BACKGROUND

Traditional mechanisms for finding healthcare services are generally manual and cumbersome. For example, a patient or a provider seeking a healthcare service such a laboratory test, an imaging scan, a specialist opinion, or other services associated with patient care and treatment, may rely on existing referral agreements (e.g., provided by a payor or by an existing referral agreement) or may manually cull bids from a variety of providers. For instance when families need to care for elderly members quite often then need to move them out of the hospital after care but are not able to take the patient home because they need skilled nursing care; finding a skilled nursing facility (SNF) that has an open bed and fits the patient needs is very difficult. Hospital social worker and family members must call each potential provider explaining what is needed and then receiving a quote back. This then needs to be cleared by any insurance carrier to then narrow the list down. All of this takes a significant amount of time; and in the process the family could lose the potential bed at the provider SNF. There are no known systems that enable high speed electronic communication between systems of different facilities for the healthcare service placement process.

Moreover, current systems do not present any such bids will not in an easy-to-ready or readily selectable format. Accordingly, an improved method for user interaction and understanding of bids for healthcare services is needed.

### SUMMARY

Embodiments of the present disclosure describe systems and methods for automatically generating and ranking search results matching a healthcare request. The provided systems use distributed computer networks across healthcare facilities, patient devices, and payor networks in combination with particular database structures and engines to allow for such automation.

In addition, the provided systems and methods may automatically assess and rank bids and display the same in an easy-to-read format along with options for selection. For example, the provided systems and methods may generate a graphical user interface with selectable bids presented in a format that facilitates prompt action by a user. Moreover, the provided systems and methods may implement a recommendation engine to provide a ranking of the selectable bids. Provided systems perform the iterative process of identifying and evaluating healthcare services for a patient via networked systems, at a speed not previously possible using traditional techniques. Disclosed techniques can prevent lost opportunities by collecting real time data from many sources quickly, and performing numerous iterations with the speed and precision necessary to identify suitable candidate services before available beds/reservations are taken. Accordingly, systems and methods of the present disclosure may improve users' experiences with systems for healthcare bids.

Furthermore, the provided systems and methods may use networked architecture to retrieve and display selectable bids faster than existing computerized technology. For example, the provided systems and methods may receive and display a plurality of bids across locations within a same short (e.g., a few seconds or a few minutes) timespan rather than pinging providers individually as existing systems do.

Disclosed embodiments included a system for automatically generating and ranking search results matching a healthcare request. The system may comprise at least one memory storing instructions and at least one processor configured to execute the instructions to: retrieve, from at least one of a consumer device or a first provider device, one or more requests, each request associated with a healthcare service and including at least one criterion associated with the service; perform at least one of: requesting one or more bids from one or more second provider devices or retrieving one or more stored bids from a database of bids, the one or more requested or retrieved bids matching the healthcare service of the one or more requests; using a recommendation engine and the at least one criterion, generate a ranking of the one or more requested or retrieved bids; generate a graphical user interface including the one or more requested or retrieved bids in a spatial order determined by the generated ranking; and output the graphical user interface to the at least one of a consumer device or a first provider device for display.

Other disclosed embodiments include a system for automatically generating and ranking search results matching a healthcare request. The system may comprise at least one memory storing instructions and at least one processor configured to execute the instructions to: retrieve, from at least one of a consumer device or a first provider device, one or more requests, each request associated with a healthcare service and including at least one criterion associated with the service; perform at least one of: requesting one or more bids from one or more second provider devices or retrieving one or more stored bids from a database of bids, the one or more requested or retrieved bids matching the healthcare service of the one or more requests; using a recommendation engine and the at least one criterion, generate a ranking of the one or more requested or retrieved bids; output the one or more requested or retrieved bids to the at least one of a consumer device or a first provider device for display in an order based on the generated ranking; receive, from the at least one of a consumer device or a first provider device, a selection of one of the one or more requested or retrieved bids; and output the selection to one of the one or more second provider devices associated with the selected one of the one or more requested or retrieved bids.

In some embodiments, the present disclosure describes non-transitory, computer-readable media for causing one or more processor to execute methods consistent with the present disclosure.

It is to be understood that the foregoing general description and the following detailed description are example and explanatory only, and are not restrictive of the disclosed embodiments.

### BRIEF DESCRIPTION OF THE DRAWINGS

The accompanying drawings, which comprise a part of this specification, illustrate several embodiments and, together with the description, serve to explain the principles disclosed herein. In the drawings:

FIG. 1 is a block diagram of a system for generating and ranking search results matching a healthcare request, according to an example embodiment of the present disclosure.

FIG. 2 is a graphical representation of an example graphical user interface (GUI) for ranking selectable bids, according to an example embodiment of the present disclosure.

FIG. 3 is a flowchart of an example method for automatically generating and ranking search results matching a healthcare request, according to an example embodiment of the present disclosure.

FIG. 4 is a flowchart of another example method for automatically generating and ranking search results matching a healthcare request, according to an example embodiment of the present disclosure.

FIG. 5 is a block diagram of an example server with which the systems, methods, and apparatuses of the present disclosure may be implemented.

FIG. 6 is a block diagram of an example patient or provider device for use in the systems, methods, and apparatuses of the present disclosure.

### DETAILED DESCRIPTION

The disclosed embodiments relate to systems and methods for automatically generating and ranking search results matching a healthcare request. Embodiments of the present disclosure may be implemented using a general-purpose computer. Alternatively, a special-purpose computer may be built according to embodiments of the present disclosure using suitable logic elements.

Advantageously, disclosed embodiments may solve the technical problem of obtaining and display selectable bids for healthcare services from a plurality of providers. As explained above, existing systems may obtain and display quotes or bids after individual pings to providers, which is slow and inefficient. Moreover, disclosed embodiments may solve the technical problems of assessing and ranking healthcare service bids. Existing systems rely on subjective and manual techniques, e.g., with using manual inputs and decision-making, thus reducing accuracy and consistency across different bidding sessions. The provided systems and methods replace such input techniques with computerized logic to increase accuracy and consistency.

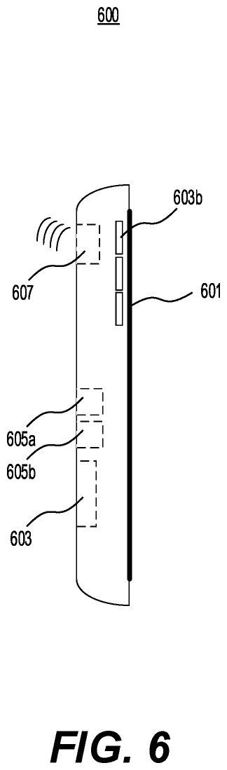

According to an aspect of the present disclosure, one or more servers or other computing devices may retrieve, from at least one of a consumer device or a first provider device, one or more requests. For example, a consumer device may comprise a smartphone (e.g., depicted in FIG. 6), a desktop, or any other computer and may be operated by a patient requesting a healthcare service (e.g., a laboratory test, an ambulance ride, a doctor's appointment, or any other healthcare service) A first provider device may comprise a smartphone (e.g., depicted in FIG. 6), a desktop, or any other computer and may be operated by a healthcare facility, such as a hospital, a doctor's office, or an outpatient facility, requesting a healthcare service from other providers. For example, the facility may request to transfer a patient to other providers, may order laboratory services or other testing from other providers, may order prescriptions from other providers, or may request any other healthcare service from other providers, e.g., on behalf of a current patient.

Each request may be associated with a healthcare service and include at least one criterion associated with the service. For example, a request may be associated with a service such as a laboratory test, an imaging scan, a specialist visit, a physical therapy visit, or other service associated with patient care or treatment. The at least one criterion may related to payment for the service (e.g., an insurance network, a copayment, coinsurance, a referral fee, or the like), quality of the service (e.g., education level of a physician, user ratings of a facility or physician, historical survey information, or the like), level of care required, length of visit or stay required, a convenience of transport to the service (e.g., based on distance, public transit options, or the like), or any other criterion relating to the healthcare service.

The requests described above may be sent over one or more computer networks, such as the Internet, a local area network (LAN), or the like, and may be sent using WiFi, a cellular or broadband network, Ethernet, or other known communication networks. In some embodiments, to retain security, the statistic(s) may be sent over a private network (such as a LAN or peer-to-peer network such as Bluetooth) and/or may be encrypted (e.g., using an Advanced Encryption Standard (AES)). In embodiments where the statistic(s) is (are) encrypted, the receiving server may decrypt the request using a private key. In embodiments where the receiving server forwards the request(s) to a different service (e.g., associated with service providers) for further processing, the receiving server may forward the encrypted request without decrypting the request first or may decrypt the request and forward the decrypted request. In embodiments where the receiving server decrypts the request, the decrypted request may be sent along a private channel (such as a private network) to the different server.

The one or more servers or other computing devices may further request one or more bids from one or more second provider devices and/or retrieve one or more stored bids from a database of bids. For example, the second provider devices may comprise a smartphone (e.g., depicted in FIG. 6), a desktop, or any other computer and may be operated by a healthcare facility, such as a hospital, a doctor's office, or an outpatient facility, requesting a healthcare service from other providers. The second providers may comprise facilities that the system selects for response to the request from a patient or from a first provider. In embodiments where the second provider devices responds to a request from a first provider device, the first and second providers may comprise separate healthcare facilities.

The one or more servers may receive bids sent from second provider devices over one or more computer networks, such as those discussed above. The one or more second provider devices may send the bids in response to receiving the request(s) form the one or more servers. Additionally or alternatively, the one or more servers may search a local database and/or a remote database for stored bids that match the requested healthcare service. For example, the database(s) may store data structures indexed by healthcare services along with elements explaining payment for the service (e.g., an insurance network, a copayment, coinsurance, a referral fee, or the like), quality of the service (e.g., education level of a physician, user ratings of a facility or physician, historical survey information, or the like), a convenience of transport to the service (e.g., based on distance, public transit options, or the like), or any other criterion relating to the healthcare service. Some elements (e.g., payment-related elements) may be provided from the one or more second provider devices and other elements (e.g., quality- or convenience-related elements or the like) may be added to the receiving server.

The one or more servers or other computing devices may use a recommendation engine and the at least one criterion to generate a ranking of the one or more requested or retrieved bids. For example, the recommendation engine may assess needs based on the request(s) (e.g., conditions of a patient associated with the request, acuity level of any such conditions, a relevant insurance network or other payment limitation such as a maximum spend, preferences stored from a requesting device, geographic limitations such as distance or transportation options, or the like) as well as capabilities of facilities associated with the bid(s) (e.g., capacities of the facilities, equipment available in the facilities, a payor network or estimated cost, historical ratings of the facilities from users and/or insurers, or the like). Accordingly, the recommendation engine may comprise a linear regression, an M2/R2 blindsolving technique, a neural network, or any other machine learning technique trained on historical data related to the associated facilities. In some embodiments, the recommendation engine may employ one or more machine learning algorithms. In some embodiments, the recommendation engine may employ one or more rule sets defining business needs for selected preferred vendors or vendors in a particular healthcare network. In some embodiments, recommendation engine may first apply the rule sets, and then provide candidates identified using the rule sets as inputs into a machine learning system.

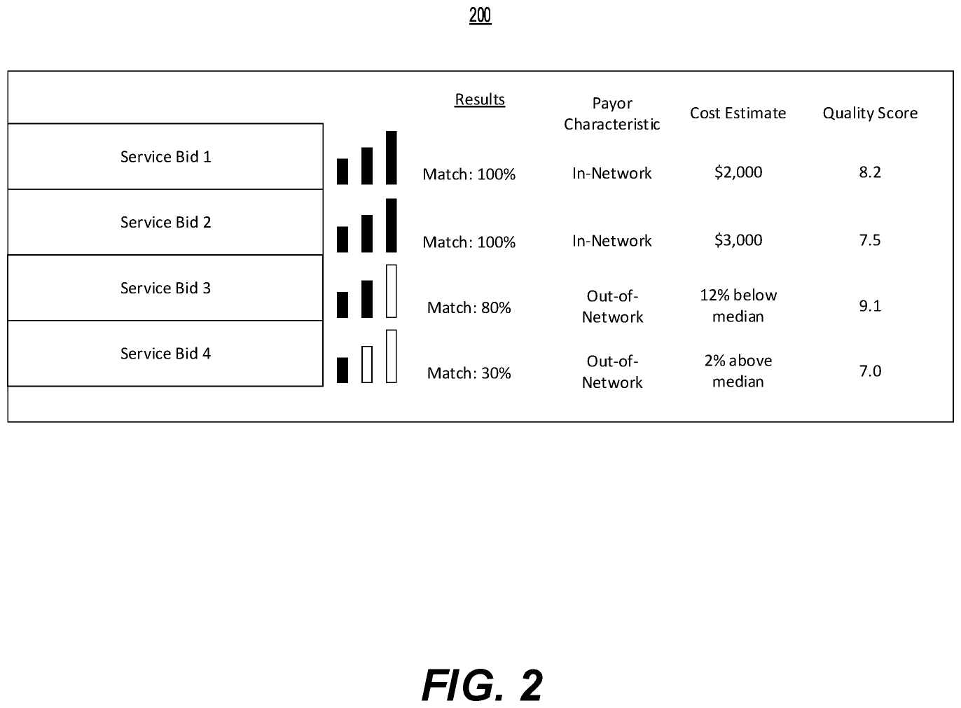

The one or more servers or other computing devices may generate a graphical user interface (GUI) including the one or more requested or retrieved bids in a spatial order determined by the generated ranking. For example, the GUI may include a ranked list of the one or more bids (e.g., as depicted in FIG. 2) and/or a graphical shape with one or more bids concentrated near a location (e.g., a center) with a distance from the location based on the ranking (not shown). One or more GUIs may also be generated to allow providers to submit bids for one or more requests, manage bids, and manage machine learning data that would be used in automated bidding processes. For example, GUIs may allow users such as vendors, social workers, or family members of patients to enter data, services provided, their location(s), references by past patients or physicians, or relationships with hospitals or other medical facilities. In some embodiments, GUIs may not be needed in systems that employ Application Programming Interfaces (APIs) such as those discussed below.

The one or more servers or other computing devices may output the graphical user interface to the at least one of a consumer device or a first provider device for display. For example, the consumer device or the first provider device may receive instructions for displaying the GUI over one or more computer networks, such as the Internet, a local area network (LAN), or the like, and using WiFi, 4G, Ethernet, or the like.

According to an aspect of the present disclosure, the one or more servers or other computing devices may output the one or more requested or retrieved bids to the at least one of a consumer device or a first provider device for display in an order based on the generated ranking, whether using a graphical user interface (GUI) or not. For example, the consumer device or the first provider device may receive an array or any other data structure storing the bids over one or more computer networks, such as the Internet, a local area network (LAN), or the like, and using WiFi, cellular or broadband network, Ethernet, or the like. In some embodiments, the consumer device or the first provider device may generate a graphical user interface (GUI) displaying the one or more bids, e.g., in a spatial order determined by the generated ranking.

The one or more servers or other computing devices may receive, from the at least one of a consumer device or a first provider device, a selection of one of the one or more requested or retrieved bids. For example, a user of the consumer device or the first provider device may use a mouse, touchscreen, keyboard, or any other input device to select one of the displayed bids. In response, the consumer device or the first provider device may transmit an indicator of the selection to the one or more servers or other computing devices over one or more computer networks, such as those previously discussed.

The one or more servers or other computing devices may further output the selection to one of the one or more second provider devices associated with the selected one of the one or more servers or other computing devices may transmit the received indicator of the selection to the one or more second provider devices over one or more computer networks, such as those discussed above.

Additionally or alternatively, the one or more servers or other computing devices may generate one or more instructions for outputting the selection to a payor system, e.g., associated with an insurer, a national system (e.g., the National Health Service (NHS) of the United Kingdom, the Centers for Medicare and Medicaid Services (CMS) of the United States, or the like), or any other entity paying for at least a portion of the requested healthcare service. Moreover, the one or more servers or other computing devices may generate and transmit one or more messages including the received indicator of the selection to the one or more second provider devices over one or more computer networks discussed above.

For example, the one or more servers or other computing devices may output a data structure, such as a portable document format (pdf) or a data structure specific to the payor system, including information used by the payor system to process payment for the service. The one or more servers or other computing devices may extract indicators of the information to include from a database of payors and corresponding to inputs to systems associated with the payors. Additionally or alternatively, the one or more servers or other computing devices may use a generic or specific template data structure and insert information from the request, the selected bid, and/or from stored information about the requester or the selected provider. For example, the one or more servers or other computing device may populate the data structure with a name of the requester, indication of the selected service, a name of the selected provider, indication of an insurance network or other relevant member of the selected provider, and any other information associated with the requester or the selected provider.

Moreover, the one or more servers or other computing devices may output a data structure, such as a message format or data structure specific to the selected provider, including information used by the selected provider to prepare for providing the requested service. For example, the one or more servers or other computing devices may activate a function provided by an application programming interface (API) of the one or more second provider devices to indicate to the one or more second provider devices that a corresponding bid has been selected. The message may include and/or API function may accept as input an identifier (e.g., a number or alphanumeric code assigned to the bid by the one or more second provider devices) of the bid being selected.

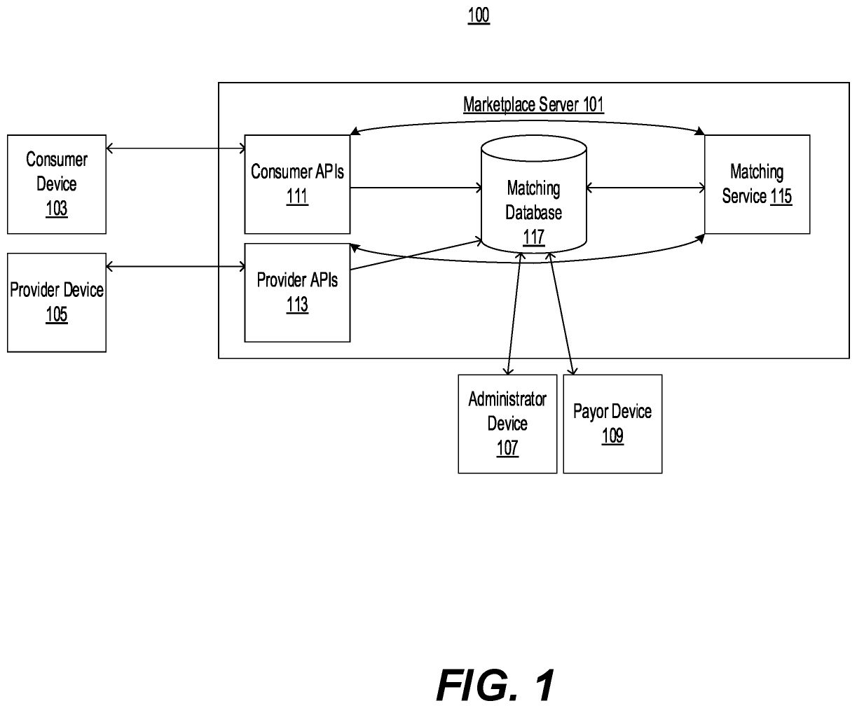

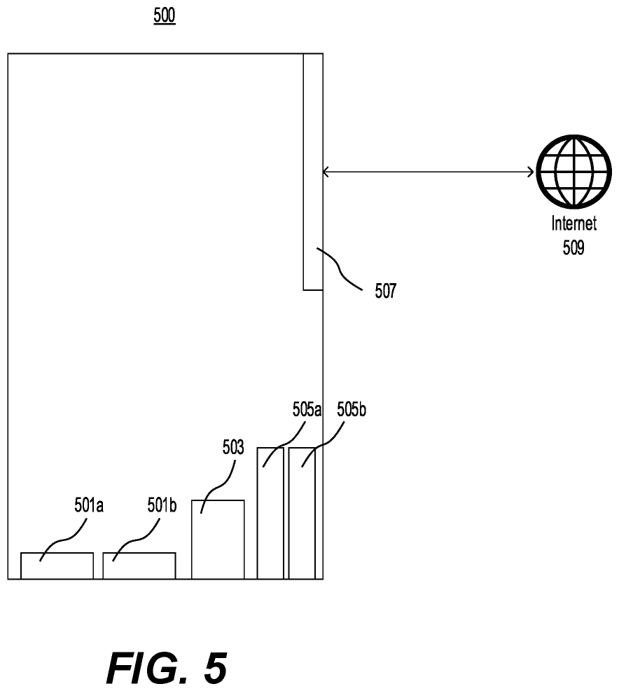

In FIG. 1, there is shown a block diagram of a system 100 for generating and ranking search results matching a healthcare request. As depicted in FIG. 1, system 100 may comprise a marketplace server 101 (e.g., comprising a computer 500 as depicted in FIG. 5 or the like) that may receive requests from consumer device 103 (e.g., comprising a computer 500 as depicted in FIG. 5, a mobile device 600 as depicted in FIG. 6, or the like) and/or provider device 105 (e.g., comprising a computer 500 as depicted in FIG. 5, a mobile device 600 as depicted in FIG. 6, or the like) using one or more APIs, e.g., consumer APIs 111 or provider APIs 113, respectively. In some embodiments, one or more of consumer APIs 111 or provider APIs 113 may require connection to a private network (and/or to a virtual private network (VPN)) for requests to be received. This may, for example, allow for requests to remain unencrypted without significant risk of interception. Additionally or alternatively, the requests may be encrypted before sending via consumer APIs 111 or provider APIs 113.

As further depicted in FIG. 1, marketplace server 101 may process requests from consumer device 103 and/or provider device 105 using a matching service 115. Accordingly, as explained above, a patient may use consumer device 103 to request bids for a healthcare service from a plurality of providers (each using provider devices 105), or one provider may use a first provider device 105 to request bids for a healthcare service from a plurality of other providers (each using second provider devices 105).

Matching service 115 may include logic executed by a processor of marketplace server 101, to obtain bids that match a healthcare service associated with the received request, e.g., from one or more providers using provider device 105 and the like. Moreover, matching service 115 may comprise a recommendation engine that uses at least one criterion to generate a ranking of the one or more requested or retrieved bids. For example, as explained above, matching service 115 may assess needs based on the request (e.g., conditions of a patient associated with the request, acuity level of any such conditions, a relevant insurance network or other payment limitation such as a maximum spend, preferences stored from a requesting device, geographic limitations such as distance or transportation options, or the like) as well as capabilities of the providers submitting the bids (e.g., capacities of the facilities of the provider, equipment available in the facilities of the provider, a payor network or estimated cost, historical ratings of the provider from users and/or insurers, or the like).

For example, matching service 115 may extract one or more of the needs from the request. Additionally or alternatively, matching service 115 may access a database with stored needs associated with indicators of healthcare services or demographics of a patient, such as an age, chronic conditions, allergies, or any other characteristics associated with the patient. In such an example, matching service 115 may extract the indicator of a healthcare service or demographics from the request and then generate a database pull using the same in order to extract one or more of the needs from the database.

Similarly, matching service 115 may extract one or more of the capabilities from the bids. Additionally or alternatively, matching service 115 may access a database with stored capabilities associated with indicators of healthcare providers, such as names, assigned identification codes, or any other alphanumeric identifier. In such an example, matching service 115 may extract the indicator of a healthcare provider from the request and then generate a database pull using the same in order to extract one or more of the capabilities from the database.

Accordingly, as explained above, matching service 115 may use a linear regression, an M2/R2 blindsolving technique, a neural network, or any other machine learning technique trained on historical data related to the associated providers.

Accordingly, in some embodiments, marketplace server 101 (e.g., comprising a computer 500 as depicted in FIG. 5 or the like) may further receive bids from provider device 105 (e.g., comprising a computer 500 as depicted in FIG. 5, a mobile device 600 as depicted in FIG. 6, or the like) using one or more APIs, e.g., consumer APIs 111 or provider APIs 113, respectively. Provider device 105 may transmit a bid in response to receiving a request from marketplace server 101. In some embodiments, one or more of provider APIs 113 may require connection to a private network (and/or to a virtual private network (VPN)) for requests to be transmitted and/or bids to be received. This may, for example, allow for requests and/or bids to remain unencrypted without significant risk of interception. Additionally or alternatively, the requests and/or bids may be encrypted before sending via one or more over provider APIs 113.

In some embodiments, provider device 105 may transmit a bid to marketplace server 101 for storage rather than in response to a request. For example, provider device 105 may generate a bid associated with a healthcare service but not with a particular patient or request. Provider device 105 may thus submit a bid for response to further requests for the healthcare service. Alternatively, provider device 105 may generate a bid associated with a particular request, and marketplace server 101 may generate a copy of the bid stripped of information related to the particular request such that the copy is associated with the requested healthcare service but not the particular request or associated patient. Thus, as explained above, marketplace server 101 may use the bid in response to future requests for the healthcare service. In any such embodiments, marketplace server 101 may use matching database 117 for indexing received bids.

Marketplace server 101 may provide the matching bids to a device associated with the request (e.g., consumer device 103 or provider device 105). For example, a data structure may be constructed or populated with the matching bids, in which the bids may be ordered according to a ranking from matching service 115. Additionally or alternatively, graphical data may be generated in conjunction with the bids to incorporate the ordered bids into an interactive GUI, e.g., as explained above and depicted in FIG. 2.

As further depicted in FIG. 1, marketplace server 101 may communicate with an administrator device 107. For example, administrator device 107 may provide new models to marketplace server 101 for use by matching service 115, tune the models used by matching service 115. Moreover, marketplace server 101 may communicator with a payor device 109. Accordingly, marketplace server 101 may receive information about payment structures from payor device 109 for use in calculating estimated costs for supplied bids from providers. Additionally or alternatively, marketplace server 101 may provide selections from consumer device 103 or provider device 105 from the ranked bids such that payor device 109 may begin processing payment for the selected provider in advance of the service being completed.

FIG. 2 shows an example graphical user interface (GUI) 200 for presenting ranking selectable bids for a healthcare service. As depicted in FIG. 2, one or more bids (e.g., service bid 1, service bid 2, service bid 3, and service bid 4) from a plurality of providers may be ordered spatially by a degree of matching with a request. Although in the example of FIG. 2 the degree of matching is depicted by bars and a percent, the degree of matching may be displayed using a letter grade, a score out of 5 or 10, a graphical element representing a degree or percentage, or any other known visual scoring mechanism.

As further depicted in FIG. 2, GUI 200 may include additional information regarding each bid, some of which may be incorporated into the degree of matching, e.g., via a recommendation engine generate the scores, as explained above. For example, as shown in FIG. 2, GUI 200 may include payor characteristics (e.g., in-network, out-of-network, or any other characteristic of the provider affecting payment for the service by the payor), cost estimates (e.g., estimated amounts total, estimated amounts out of pocket if a payor is covering part, estimated amounts above or below a median of the bids, or the like), and a quality score (e.g., based on historical reviews of the providers, quality scores developed by the payor, or the like). Additional or alternative information may be shown, such as a distance to the provider from a current location of a requester, a current capacity of the provider, or the like.

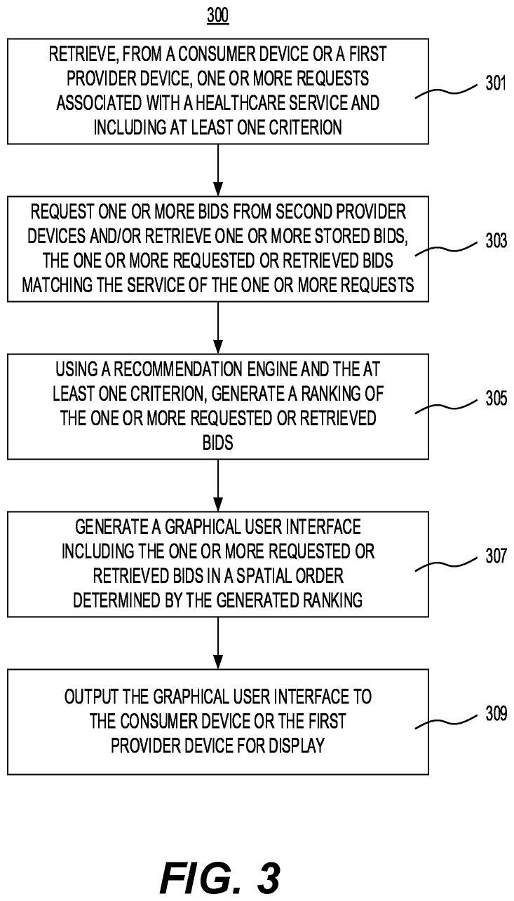

FIG. 3 depicts an example method 300 for automatically generating and ranking search results matching a healthcare request. Method 300 may be implemented using one or more processor (e.g., processor 503 of FIG. 5).

At step 301, the processor may retrieve, from at least one of a consumer device (e.g., consumer device 103 of FIG. 1) or a first provider device (e.g., provider device 105 of FIG. 1), one or more requests. As explained above, a patient may submit a request for bids (e.g., using consumer device 103) for a healthcare service from a plurality of providers (e.g., using provider devices 105), or one provider submit a request for bids (e.g., using a first provider device 105) for a healthcare service from a plurality of other providers (e.g., using second provider devices 105).

As further explained above, each request may be associated with a healthcare service and include at least one criterion associated with the service. Moreover, the one or more requests may be sent to the marketplace system (e.g., marketplace server 101 of FIG. 1) over one or more computer networks, such as those previously discussed. In some embodiments, to retain security, the one or more requests may be sent over a private network (such as a LAN) or a peer-to-peer connection, and/or may be encrypted (e.g., using an Advanced Encryption Standard (AES)).

At step 303, the processor may perform at least one of: requesting one or more bids from one or more second provider devices or retrieving one or more stored bids from a database of bids, the one or more requested or retrieved bids matching the healthcare service of the one or more requests. For example, as explained above, the marketplace system (such as marketplace server 101 of FIG. 1) may send the request(s) to provider devices (e.g., provider device 105 of FIG. 1). Moreover, the marketplace system may receive the bid(s) in response to the sent request(s). Additionally or alternatively, the marketplace system may obtain bid(s) from a database (e.g., matching database 117 of FIG. 1) of bids previously received from provider devices (e.g., provider device 105 of FIG. 1). The processor may receive the bid(s) via the same API(s) as the request(s) and/or via one or more different APIs. In some embodiments, the system may employ a set of APIs, and may adhere to open API standards such as the OpenAPI Specification.

At step 305, the processor, may using a recommendation engine and the at least one criterion (discussed in further detail below), generate a ranking of the one or more requested or retrieved bids. For example, as explained above, the processor may assess needs based on the request (e.g., conditions of a patient associated with the request, acuity level of any such conditions, a relevant insurance network or other payment limitation such as a maximum spend, preferences stored from a requesting device, geographic limitations such as distance or transportation options, or the like) as well as capabilities of providers submitting the bids (e.g., capacities of the facilities of the provider, equipment available in the facilities of the provider, a payor network or estimated cost, historical ratings of the provider from users and/or insurers, or the like). Accordingly, as explained above, the processor may use a linear regression, an M2/R2 blindsolving technique, a neural network, or any other machine learning technique trained on historical data related to the associated providers.

At step 307, the processor may generate a graphical user interface including the one or more requested or retrieved bids in a spatial order determined by the generated ranking. For example, as explained above, the processor may generate GUI 200 of FIG. 2 or any other GUI including the bid(s) based on the generated ranking.

At step 309, the processor may generate instructions to output the graphical user interface to the at least one of a consumer device or a first provider device for display. For example, the processor may transmit data associated with the GUI to a device associated with the requesting party (e.g., consumer device 103 or provider device 105 of FIG. 1), e.g., over a secure connection and/or after encrypting of the GUI.

Method 300 may include additional steps. For example, in some embodiments method 300 may further include storing received bids in a database (e.g., matching database 117 of FIG. 1) and indexing the same by associated healthcare services.

In any of the embodiments described above, the processor may calculate one or more criteria to associate with the bids before generating a GUI in step 307 and/or storing the bids in the database. For example, as explained above, some criteria (e.g., payment-related elements) may be provided from the one or more second provider devices but other criteria (e.g., quality- or convenience-related elements of the like) may be added by the processor. In some embodiments, data for the quality of a provider may include patient opinions on care received, outcome of results from services provided, external rating services for a provider, years of service of the provider, and other objective or subjective metrics pertinent to the quality of the provider.

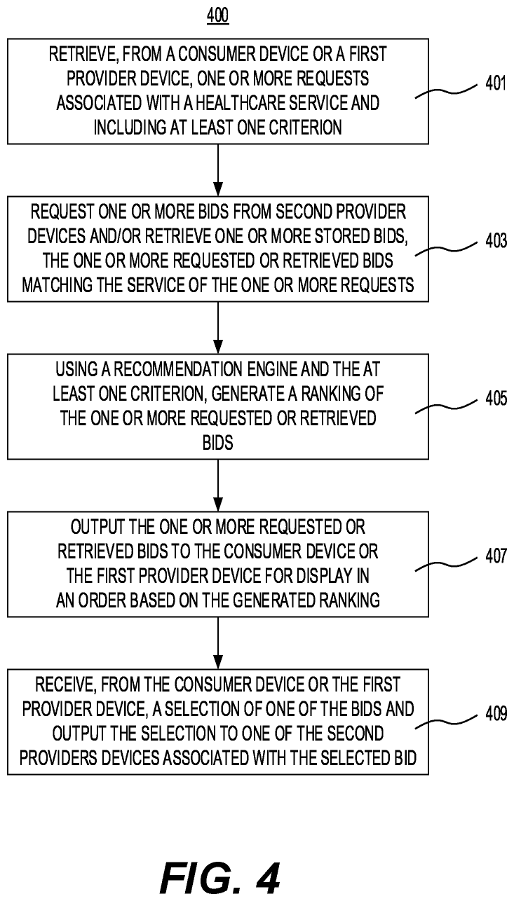

FIG. 4 depicts an example method 400 for automatically generating and ranking search results matching a healthcare request. Method 400 may be implemented using one or more processor (e.g., processor 503 of FIG. 5).

Steps 401, 403, and 405 may be executed similarly to steps 301, 303, and 305 of FIG. 3, described above.

At step 407, the processor may generate data or instructions for outputting the one or more requested or retrieved bids to the at least one of a consumer device or a first provider device for display in an order based on the generated ranking. For example, as explained above, the processor may generate an array or other data structure with the ranked bids, and the receiving device may display the array or other data structure to a user of said device.

At step 409, the processor may receive, from the at least one of a consumer device or a first provider device, a selection of one of the one or more requested or retrieved bids via input received using an interactive graphical user interface, and output the selection to one of the one or more second provider devices associated with the selected one of the one or more requested or retrieved bids. For example, as explained above, marketplace server 101 may receive an encrypted data structure indicating the selection and similarly transmit an encrypted data structure (whether encrypted the same way or a different way) including the selection.

Method 400 may include additional steps. For example, method 400 may further transmit the selection to a device associated with a payor (e.g., payor device 109 of FIG. 1). Accordingly, payor device 109 may process payment based on the data structure received from the marketplace server 101.

FIG. 5 is block diagram of an example device 500 suitable for implementing the disclosed systems and methods. For example, device 500 may execute method 300 of FIG. 3 and/or method 400 of FIG. 4. Device 500 may comprise a server, desktop computer, or another computerized device capable of performing the functions disclosed herein. In some embodiments, device 500 may be comprised of a plurality of distributed computing devices, or a cloud computing system. Device 500 may comprise marketplace server 101 of FIG. 1 or in any other system configured to rank matching healthcare service bids.

As depicted in FIG. 5, example server 500 may include at least one processor (e.g., processor 503) and at least one memory (e.g., memories 505A and 505b).

Processor 503 may comprise a central processing unit (CPU), a graphics processing unit (GPU), or other similar circuitry capable of performing one or more operations on a data stream. Processor 503 may comprise a single or multicore processor, or a collection of processor in communication to perform distributed computing functions. Processor 503 may be configured to execute instructions that may, for example, be stored on one or more of memories 505a and 5050b.

Memories 505a and 505b may be volatile memory (such as RAM or the like) and/or non-volatile memory (such as flash memory, a hard disk drive, or the like). In some embodiments, memories 505a and 5050b may comprise distributed data storage devices in communication to support a cloud computing system. As explained above, memories 505a and 505b may store instructions for execution by processor 503.

As further depicted in FIG. 5, server 500 may include at least one network interface controller (NIC) (e.g., NIC 507). NIC 507 may be configured to facilitated communication over at least one computing network (e.g., network 509, which is depicted in the example of FIG. 5 as the Internet). Communication functions may thus be facilitated through one or more NICs, which may be wireless and/or wired and may include an Ethernet port, radio frequency receivers and transmitters, and/or optical (e.g., infrared) receivers and transmitters. The specific design and implementation of the one or more NICs depend on the computing network 509 over which server 500 is intended to operate. For example, in some embodiments, server 500 may include one or more wireless and/or wired NICs designed to operate over a GSM network, a GPRS network, an EDGE network, a Wi-Fi or WiMax network, and a Bluetooth® network. Alternatively or concurrently, server 500 may include one or more wireless and/or wired NICs designed to operate over a TCP/IP network.

As depicted in FIG. 5, server 500 may include and/or be operably connected to one or more storage devices, e.g., storages 501a and 501b. Storage devices 501a and 501b may be volatile (such as RAM or the like) or non-volatile (such as flash memory, a hard disk drive, or the like). In some embodiments, storages 501a and 501b may comprise distributed data storage devices in communication to support a cloud computing system.

Processor 502, memories 505a and 505b, NIC 507, and/or storage devices 501a and 501b may comprise separate components or may be integrated in one or more integrated circuits. The various components in server 500 may be coupled by one or more communication buses or signal lines (not shown).

FIG. 6 is a block diagram of an example mobile device 600 for collecting requests or bids for a marketplace system (e.g., marketplace server 101 of FIG. 1, which may comprise server 500 of FIG. 5). Device 600 may comprise a smartphone, a tablet, a portable computer, a body-worn computing device such as a smartwatch, or another computing device capable of performing techniques disclosed herein.

Device 600 may have a screen 601. For example, screen 601 may display one or more graphical user interfaces (GUIs). In certain aspects, screen 601 may comprise a touchscreen to facilitate use of the one or more GUIs.

As further depicted in FIG. 6, mobile device 600 may have at least one processor 603. For example, at least one processor 603 may comprise a system-on-a-chip (SOC) adapted for use in a portable device, such as device 600. Alternatively or concurrently, at least one processor 603 may comprise any other type(s) of processor.

As further depicted in FIG. 6, mobile device 600 may have one or more memories, e.g., memories 605a and 605b. In certain aspects, some of the one or more memories, e.g., memory 605a, may comprise a volatile memory. In such aspects, memory 605a, for example, may store one or more applications (or “apps”) for execution on at least one processor 603. For example, an app may include an operating system for intake device 600 and/or an app for collecting input to encode as a request or bid, providing an application programming interface (API) to one or more authorized marketplace systems (e.g., marketplace server 101 of FIG. 1), and/or displaying a GUI (e.g., GUI 200 of FIG. 2) with selectable bid results. In addition, memory 605a may store data generated by, associated with, or otherwise unrelated to an app in memory 605a.

Alternatively or concurrently, some of the one or more memories, e.g., memory 605b, may comprise a non-volatile memory. In such aspects, memory 605b, for example, may store one or more applications (or “apps”) for execution on at least one processor 603. For example, as discussed above, an app may include an operating system for intake device 600 and/or an app as explained above. In addition, memory 605b may store data generated by, associated with, or otherwise unrelated to an app in memory 605b. Furthermore, memory 605b may include a pagefile, swap partition, or other allocation of storage to allow for the use of memory 605b as a substitute for a volatile memory if, for example, memory 605a is full or nearing capacity.

As depicted in FIG. 6, mobile device 600 may include at least one network interface controller (NIC) (e.g., NIC 607). NIC 607 may be configured to facilitate communication over at least one computing network. Communication functions may thus be facilitated through one or more NICs. Although depicted in wireless in FIG. 6 and including radio frequency receivers and transmitters and/or optical (e.g., infrared) receiver and transmitters, NIC 607 may alternatively be wired and include an Ethernet port or the like. The specific design and implementation of the one or more NICs depend on the computer network over which mobile device 600 is intended to operate. For example, in some embodiments, mobile device 600 may include one or more wireless and/or wired NICs designed to operate over a GSM network, a GPRS network, an EDGE network, a Wi-Fi or WiMax network, and a Bluetooth® network. Alternatively or concurrently, mobile device 600 may include one or more wireless and/or wired NICs designed to operator over a TCP/IP network.

Each of the above identified instructions and application may correspond to a set of instructions for performing one or more functions described above. These instructions need not be implemented a separate software programs, procedures, or modules. Disclosed memories may include additional instructions or fewer instructions. Furthermore, device 600 may securely deliver requests and/or bids to server 500 (which may, for example, comprise marketplace server 101 of FIG. 1). These functions of device 600 may be implemented in hardware and/or in software, including in one or more signal processing and/or application specific integrated circuits.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. For example, the described implementations include hardware and software, but systems and methods consistent with the present disclosure can be implemented with hardware alone. In addition, while certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations and/or alterations based on the present disclosure. The elements in the claims are to be interpreted broadly based on the language employed in the claims and not limited to examples described in the present specification or during the prosecution of the application, which examples are to be construed as nonexclusive.

Instructions or operational steps stored by a computer-readable medium may be in the form of computer programs, program modules, or codes. As described herein, computer programs, program modules, and code based on the written description of this specification, such as those used by the processor, are readily within the purview of a software developer. The computer programs, program modules, or code can be created using a variety of programming techniques. For example, they can be designed in or by means of Java, C, C++, assembly language, or any such programming languages. One or more of such programs, modules, or code can be integrated into a device system or existing communications software. The programs, modules, or code can also be implemented or replicated as firmware or circuit logic.

The features and advantages of the disclosure are apparent from the detailed specification, and thus, it is intended that the appended claims cover all systems and methods failing within the true spirit and scope of the disclosure. As used herein, the indefinite articles “a” and “an” mean “one or more.” Similarly, the use of a plurality term does not necessarily denote a plurality unless it is unambiguous in the given context. Words such as “and” or “or” mean “and/or” unless specifically directed otherwise. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

Other embodiments will be apparent from consideration of the specification and practice of the embodiments disclosed herein. It is intended that the specification and examples be considered as example only, with a true scope and spirit of the disclosed embodiments being indicated by the following claims.

What is claimed is:

1. A system for automatically generating and ranking search results matching a healthcare request, the system comprising:
at least one memory storing instructions; and
at least one processor configured to execute the instructions to:
retrieve, from at least one of a consumer device or a first provider device, one or more requests, each request associated with a healthcare service and including at least one criterion associated with the service;
perform at least one of: requesting one or more bids from one or more second provider devices or retrieving one or more stored bids from a database of bids, the one or more requested or retrieved bids matching the healthcare service of the one or more requests;
using a recommendation engine and the at least one criterion, generate a ranking of the one or more requested or retrieved bids;
generate a graphical user interface including the one or more requested or retrieved bids in a spatial order determined by the generated ranking; and
output the graphical user interface to the at least one of a consumer device or a first provider device for display.

2. The system of claim 1, wherein the one or more requests are encrypted and wherein the retrieving comprises decrypting the encrypted one or more requests.

3. The system of claim 1, wherein the retrieving one or more stored bids comprises searching the database for stored bids matching the service.

4. The system of claim 1, wherein the generating a ranking comprises assessing needs based upon one or more requests and capabilities of facilities corresponding to the one or more second provider devices and associated with the one or more bids.

5. The system of claim 1, wherein the recommendation engine comprises a machine learning technique trained on historical data related to the facilities corresponding to the one or more second provider devices.

6. The system of claim 1, wherein the recommendation engine employs at least one of: one or more machine learning algorithms and one or more rule sets defining business needs for selecting a preferred vendor.

7. The system of claim 1, wherein the performing comprises obtaining, using a matching service, bids that match the healthcare service.

8. The system of claim 7, wherein the obtaining comprises extracting one or more needs from the request and obtaining bids that match the one or more needs.

9. The system of claim 1, wherein the spatial order is determined by a degree of matching of the one or more requested or retrieved bids to the one or more requests.

10. The system of claim 1, wherein the at least one criterion is selected from the group consisting of: payment for the service, quality of the service, level of care requested, length of visit required, and transport to the service.

11. A system for automatically generating and ranking search results matching a healthcare request, the system comprising:
at least one memory storing instructions; and
at least one processor configured to execute the instructions to:
retrieve, from at least one of a consumer device or a first provider device, one or more requests, each request associated with a healthcare service and including at least one criterion associated with the service;
perform at least one of: requesting one or more bids from one or more second provider devices or retrieving one or more stored bids from a database of bids, the one or more requested or retrieved bids matching the healthcare service of the one or more requests;
using a recommendation engine and the at least one criterion, generate a ranking of the one or more requested or retrieved bids;
output the one or more requested or retrieved bids to the at least one of a consumer device or a first provider device for display in an order based on the generated ranking;
receive, from the at least one of a consumer device or a first provider device, a selection of one of the one or more requested or retrieved bids; and
output the selection to one of the one or more second provider devices associated with the selected one of the one or more requested or retrieved bids.

12. The system of claim 11, wherein the one or more requests are encrypted and wherein the retrieving comprises decrypting the encrypted one or more requests.

13. The system of claim 11, wherein the retrieving one or more stored bids comprises searching the database for stored bids matching the service.

14. The system of claim 11, wherein the generating a ranking comprises assessing needs based upon one or more requests and capabilities of facilities corresponding to the one or more second provider devices and associated with the one or more bids.

15. The system of claim 11, wherein the recommendation engine comprises a machine learning technique trained on historical data related to the facilities corresponding to the one or more second provider devices.

16. The system of claim 11, wherein the recommendation engine employs at least one of: one or more machine learning algorithms and one or more rule sets defining business needs for selecting a preferred vendor.

17. The system of claim 11, wherein the performing comprises obtaining, using a matching service, bids that match the healthcare service.

18. The system of claim 17, wherein the obtaining comprises extracting one or more needs from the request and obtaining bids that match the one or more needs.

19. The system of claim 11, wherein the order is determined by a degree of matching of the one or more requested or retrieved bids to the one or more requests.

20. The system of claim 11, wherein the at least one criterion is selected from the group consisting of: payment for the service, quality of the service, level of care requested, length of visit required, and transport to the service.
